(19) United States
(12) Patent Application Publication
(10) Pub. No.: US 2024/0038395 A1
(43) Pub. Date: Feb. 1, 2024

(54) MACHINE LEARNING-BASED PREDICTION OF TREATMENT REQUIREMENTS FOR NEOVASCULAR AGE-RELATED MACULAR DEGENERATION (NAMD)

(71) Applicants: Genentech, Inc., South San Francisco, CA (US); Hoffmann La-Roche Inc., Little Falls, NJ (US)

(72) Inventors: Andreas MAUNZ, Freiburg (DE); Ales NEUBERT, Mendrisio (CH); Andreas THALHAMMER, Basel (CH); Jian DAI, Fremont, CA (US)

(21) Appl. No.: 18/482,264

(22) Filed: Oct. 6, 2023

Related U.S. Application Data
(63) Continuation of application No. PCT/US2022/023937, filed on Apr. 7, 2022.
(60) Provisional application No. 63/172,082, filed on Apr. 7, 2021.

(51) Int. Cl. G16H 50/20; A61B 3/12; A61B 3/00; G06T 7/00; G06T 7/11

(52) U.S. Cl. CPC G16H 50/20; A61B 3/1225; A61B 3/0025; G06T 7/0016; G06T 7/11; A61B 3/102

(57) ABSTRACT

A method and system for managing a treatment for a subject diagnosed with neovascular age-related macular degeneration (nAMD). Spectral domain optical coherence tomography (SD-OCT) imaging data of a retina of the subject is received. Retinal feature data is extracted for a plurality of retinal features using the SD-OCT imaging data, the plurality of retinal features being associated with at least one of a set of retinal fluids or a set of retinal layers. Input data formed using the retinal feature data for the plurality of retinal features is sent into a first machine learning model. A treatment level for an anti-vascular endothelial growth factor (anti-VEGF) treatment to be administered to the subject is predicted, via the first machine learning model, based on the input data.

### CROSS-REFERENCE TO RELATED APPLICATIONS

This application is a continuation of International Application No. PCT/US2022/023937, filed Apr. 7, 2022, and entitled “Machine Learning-Based Prediction of Treatment Requirements for Neovascular Age-Related Macular Degeneration (nAMD),” which claims priority to U.S. Provisional Patent Application No. 63/172,082, filed Apr. 7, 2021, and entitled “Machine Learning-Based Prediction of Treatment Requirements for Neovascular Age-Related Macular Degeneration (nAMD),” which are incorporated herein by reference in their entirety.

### FIELD

This application relates to treatment requirements for neovascular age-related macular degeneration (nAMD), and more particularly, to machine learning-based prediction of treatment requirements in nAMD using spectral domain optical coherence tomography (SD-OCT).

### BACKGROUND

Age-related macular degeneration (AMI)) is a leading cause of vision loss in subjects 50 years and older. AMD initially manifests as a dry type of AMD and progresses to a wet type of AMD, also referred to as neovascular AMD (nAMD). For the dry type, small deposits (drusen) form under the macula on the retina, causing the retina to deteriorate in time. For the wet type, abnormal blood vessels originating in the choroid layer of the eye grow into the retina and leak fluid from the blood into the retina. Upon entering the retina, the fluid may distort the vision of a subject immediately, and over time, can damage the retina itself, for example, by causing the loss of photoreceptors in the retina. The fluid can cause the macula to separate from its base, resulting in severe and fast vision loss.

Anti-vascular endothelial growth factor (anti-VEGF) agents are frequently used to treat the wet type of AMD (or nAMD). Specifically, anti-VEGF agent can dry out a subject's retina, such that the subject's wet type of AMD can be better controlled to reduce or prevent permanent vision loss. Anti-VEGF agents are typically administered via intravitreal injections, which are both disfavored by subjects and can be accompanied by side effects (e.g., red eye, sore eye, infection, etc.). The number or frequency of the injections can also be burdensome on patients and lead to decreased control of the disease.

### SUMMARY

In one or more embodiments, a method is provided for managing a treatment for a subject diagnosed with neovascular age-related macular degeneration (nAMD). Spectral domain optical coherence tomography (SD-OCT) imaging data of a retina of the subject is received. Retinal feature data is extracted for a plurality of retinal features using the SD-OCT imaging data, the plurality of retinal features being associated with at least one of a set of retinal fluids or a set of retinal layers. Input data formed using the retinal feature data for the plurality of retinal features is sent into a first machine learning model. A treatment level for an anti-vascular endothelial growth factor (anti-VEGF) treatment to be administered to the subject is predicted, via the first machine learning model, based on the input data.

In one or more embodiments, a method is provided for managing an anti-vascular endothelial growth factor (anti-VEGF) treatment for a subject diagnosed with neovascular age-related macular degeneration (nAMD). A machine learning model is trained using training input data to predict a treatment level for the anti-VEGF treatment, wherein the training input data is formed using training optical coherence tomography (OCT) imaging data. Input data is received for the trained machine learning model, the input data comprising retinal feature data for a plurality of retinal features. The treatment level for the anti-VEGF treatment to be administered to the subject is predicted, via the trained machine learning model, using the input data.

In one or more embodiments, a system for managing an anti-vascular endothelial growth factor (anti-VEGF) treatment for a subject diagnosed with neovascular age-related macular degeneration (nAMD) comprises a memory containing machine readable medium comprising machine executable code and a processor coupled to the memory. The processor is configured to execute the machine executable code to cause the processor to: receive spectral domain optical coherence tomography (SD-OCT) imaging data of a retina of the subject; extract retinal feature data for a plurality of retinal features using the SD-OCT imaging data, the plurality of retinal features being associated with at least one of a set of retinal fluids or a set of retinal layers; send input data formed using the retinal feature data for the plurality of retinal features into a first machine learning model; and predict, via the first machine learning model, a treatment level for an anti-vascular endothelial growth factor (anti-VEGF) treatment to be administered to the subject based on the input data.

In some embodiments, a system is provided that includes one or more data processors and a non-transitory computer readable storage medium containing instructions which, when executed on the one or more data processors, cause the one or more data processors to perform part or all of one or more methods disclosed herein.

In some embodiments, a computer-program product is provided that is tangibly embodied in a non-transitory machine-readable storage medium and that includes instructions configured to cause one or more data processors to perform part or all of one or more methods disclosed herein.

Some embodiments of the present disclosure include a system including one or more data processors. In some embodiments, the system includes a non-transitory computer readable storage medium containing instructions which, when executed on the one or more data processors, cause the one or more data processors to perform part or all of one or more methods and/or part or all of one or more processes disclosed herein. Some embodiments of the present disclosure include a computer-program product tangibly embodied in a non-transitory machine-readable storage medium, including instructions configured to cause one or more data processors to perform part or all of one or more methods and/or part or all of one or more processes disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed Thus, it should be understood that although the present invention as claimed has been specifically disclosed by embodiments and optional features, modification and variation of the concepts herein disclosed may be resorted to by those skilled in the art, and that such modifications and variations are considered to be within the scope of this invention as defined by the appended claims.

### BRIEF DESCRIPTION OF THE DRAWINGS

For a more complete understanding of the principles disclosed herein, and the advantages thereof, reference is now made to the following descriptions taken in conjunction with the accompanying drawings, in which:

FIG. 1 is a block diagram of a treatment management system in accordance with one or more embodiments.

FIG. 2 is a block diagram of the treatment level prediction system from FIG. 1 being used in a training mode in accordance with one or more embodiments.

FIG. 3 is a flowchart of a process for managing a treatment for a subject diagnosed with nAMD in accordance with one or more embodiments.

FIG. 4 is a flowchart of a process for managing a treatment for a subject diagnosed with nAMD in accordance with one or more embodiments.

FIG. 5 is a flowchart of a process for managing a treatment for a subject diagnosed with nAMD in accordance with one or more embodiments.

FIG. 6 is an illustration of a segmented OCT image in accordance with one or more embodiments.

FIG. 7 is an illustration of a segmented OCT image in accordance with one or more embodiments.

FIG. 8 is a plot illustrating the results of a 5-fold cross-validation for a treatment level classification of “low” in accordance with one or more embodiments.

FIG. 9 is a plot illustrating the results of a 5-fold cross-validation for a treatment level classification of “high” in accordance with one or more embodiments.

FIG. 10 is a plot of AUC data illustrating the results of repeated 5-fold cross-validation for a treatment level classification of “high” in accordance with one or more embodiments.

FIG. 11 is a block diagram illustrating an example of a computer system in accordance with one or more embodiments.

It is to be understood that the figures are not necessarily drawn to scale, nor are the objects in the figures necessarily drawn to scale in relationship to one another. The figures are depictions that are intended to bring clarity and understanding to various embodiments of apparatuses, systems, and methods disclosed herein. Wherever possible, the same reference numbers will be used throughout the drawings to refer to the same or like parts. Moreover, it should be appreciated that the drawings are not intended to limit the scope of the present teachings in any way.

### DETAILED DESCRIPTION

### I. Overview

Neovascular age-related macular degeneration (nAMD) may be treated with anti-vascular endothelial growth factor (anti-VEGF) agents that are designed to treat nAMD by drying out the retina of a subject to avoid or reduce permanent vision loss. Examples of anti-VEGF agents include ranibizumab and aflibercept. Typically, anti-VEGF agents are administered via intravitreal injection at a frequency ranging from about every four weeks to about eight weeks. Some patients, however, may not require such frequent injections.

The frequency of the treatments may be generally burdensome to patients and may contribute to decreased disease control in the real-world. For example, after an initial phase of treatment, patients may be scheduled for regular monthly visits over a pro re nata (PRN) or as needed period of time. This PRN period of time may be, for example, 21 to 24 months, or some other number of months. Traveling to a clinic for monthly visits during the PRN period of time may be burdensome for patients who do not need frequent treatments. For example, it may be overly burdensome to travel for monthly visits when the patient will only need 5 or fewer injections during the entire PRN period. Accordingly, patient compliance with visits may decrease over time, leading to reduced disease control.

Thus, there is a need for methods and systems that allow for predicting anti-VEGF treatment requirements to help guide and ensure effective treatment of nAMD patients with injections of anti-VEGF agents. The embodiments described herein provide methods and systems for predicting a treatment level that will be needed for patients.

Some patients may have “low” treatment needs or requirements while others may have “high” treatment needs or requirements. The thresholds for defining these treatment levels (i.e., “low” or “high” treatment level) may be based on the number of anti-VEGF injections and the time period during which the injections are administered. For example, a patient that receives 8 or fewer anti-VEGF injections over a 24-month period may be considered as having a “low” treatment level. For instance, the patient may receive monthly anti-VEGF injections for three months and receive five or fewer anti-VEGF injections over the PRN period of 21 months. On the other hand, a patient that receives 19 or more anti-VEGF injections over a 24-month period may be considered as belonging in the group of patients having a “high” treatment level. For instance, the patient may receive monthly anti-VEGF injections for three months and receive 16 or more injections over the PRN period of 21 months.

Additionally, other treatment levels may be evaluated, such as, for example, a “moderate” treatment level (e.g., 9-18 injections over 24-month period) indicating a treatment requirement between “low” and “high” treatment needs or requirements. The frequency of injections administered to a patient may be based on what is needed to effectively reduce or prevent ophthalmic complications of nAMD, such as, but not limited to, leakage of blood vessel fluids into a retina, etc.

The embodiments described herein use machine learning models to predict treatment level. In one or more embodiments, spectral domain optical coherence tomography (SD-OCT) images of the eyes of subjects with nAMD may be obtained. OCT is an imaging technique in which light is directed at a biological sample (e.g., biological tissue such as an eye) and the light that is reflected from features of that biological sample is collected to capture two-dimensional or three-dimensional, high-resolution cross-sectional images of the biological sample. In SD-OCT, also known as Fourier domain OCT, signals are detected as a function of optical frequencies (e.g., in contrast to as a function of time).

The SD-OCT images may be processed using a machine learning (ML) model (e.g., a deep learning model) that is configured to automatically segment the SD-OCT images and generate segmented images. These segmented images identify one or more retinal fluids, one or more retinal layers, or both, on the pixel level. Quantitative retinal feature data may then be extracted from these segmented images. In one or more embodiments, the machine learning model is trained for both segmentation and feature extraction.

A retinal feature may be associated with one or more retinal pathologies (e.g., retinal fluids), one or more retina layers, or both. Examples of retinal fluids include, but are not limited to, an intraretinal fluid (IRF), a subretinal fluid (SRF), a fluid associated with pigment epithelial detachment (PED), and a subretinal hyperreflective material (SHRM). Examples of retinal layers include, but are not limited to, an internal limiting membrane (ILM) layer, an outer plexiform layer-Henle fiber layer (OPL-HFL), an inner boundary-retinal pigment epithelial detachment (IB-RPE), an outer boundary-retinal pigment epithelial detachment (OB-RPE), and a Bruch's membrane (BM).

The embodiments described herein may use another machine learning model (e.g., a symbolic model) to process the retinal feature data (e.g.. some or all of the retinal feature data extracted from the segmented images) and predict the treatment level (e.g., a classification for the treatment level). Different retinal features may have varying levels of importance to the predicted treatment level. For example, one or more features associated with PED during an early stage of anti-VEGF treatment (e.g., at the second month of anti-VEGF treatment during the afore-mentioned 24-month treatment schedule) may be strongly associated with a low treatment level during the PRN phase. As another example, one or more features associated with SHRM during an early stage of anti-VEGF treatment (e.g., at the first month of anti-VEGF treatment during the 24-month treatment schedule) may be strongly associated with a high treatment level.

With the predicted treatment level, an output (e.g., report) can be generated that will help guide overall treatment management. For example, when the predicted treatment level is high, the output may identify a set of strict protocols that can be put in place to ensure patient compliance with clinic visits. When the predicted treatment level is low, the output may identify a more relaxed set of protocols that can be put in place to reduce the burden on the patient. For example, rather than the patient having to travel for monthly clinic visits, the output may identify that the patient can be evaluated at the clinic every two or three months.

Using the automatically segmented images generated by a machine learning model (e.g., deep learning model) to automatically extract the retinal feature data for use in predicting treatment level via another machine learning model (e.g., symbolic model) may reduce the overall computing resources and/or time needed to predict treatment level and may ensure improved accuracy of the predicted treatment level. Using these methods may improve the efficiency of predicting treatment level. Further, being able to accurately and efficiently predict treatment level may help with overall nAMD treatment management in reducing the overall burden felt by nAMD patients.

Recognizing and taking into account the importance and utility of a methodology and system that can provide the improvements described above, the embodiments described herein enable predicting treatment requirements for nAMD with anti-VEGF agent injections. More particularly, the embodiments described herein use SD-OCT and ML-based predictive modeling to predict anti-VEGF treatment requirements for patients with nAMD.

### II. Exemplary Definitions and Context

The disclosure is not limited to these exemplary embodiments and applications or to the manner in which the exemplary embodiments and applications operate or are described herein. Moreover, the figures may show simplified or partial views, and the dimensions of elements in the figures may be exaggerated or otherwise not in proportion.

In addition, as the terms “on,” “attached to,” “connected to,” “coupled to,” or similar words are used herein, one element (e.g., a component, a material, a layer, a substrate, etc.) can be “on,” “attached to,” “connected to,” or “coupled to” another element regardless of whether the one element is directly on, attached to, connected to, or coupled to the other element or there are one or more intervening elements between the one element and the other element. In addition, where reference is made to a list of elements (e.g., elements a, b, c), such reference is intended to include any one of the listed elements by itself, any combination of less than all of the listed elements, and/or a combination of all of the listed elements. Section divisions in the specification are for ease of review only and do not limit any combination of elements discussed.

The term “subject” may refer to a subject of a clinical trial, a person undergoing treatment, a person undergoing anti-cancer therapies, a person being monitored for remission or recovery, a person undergoing a preventative health analysis (e.g., due to their medical history), or any other person or subject of interest. In various cases, “subject” and “subject” may be used interchangeably herein. In various cases, a “subject” may also be referred to as a “patient”.

Unless otherwise defined, scientific and technical terms used in connection with the present teachings described herein shall have the meanings that are commonly understood by those of ordinary skill in the art. Further, unless otherwise required by context, singular terms shall include pluralities and plural terms shall include the singular. Generally, nomenclatures utilized in connection with, and techniques of, chemistry, biochemistry, molecular biology, pharmacology and toxicology are described herein are those well-known and commonly used in the art.

As used herein, “substantially” means sufficient to work for the intended purpose. The term “substantially” thus allows for minor, insignificant variations from an absolute or perfect state, dimension, measurement, result, or the like such as would be expected by a person of ordinary skill in the field but that do not appreciably affect overall performance. When used with respect to numerical values or parameters or characteristics that can be expressed as numerical values, “substantially” means within ten percent.

As used herein, the term “about” used with respect to numerical values or parameters or characteristics that can be expressed as numerical values means within ten percent of the numerical values. For example, “about 50” means a value in the range from 45 to 55, inclusive.

The term “ones” means more than one.

As used herein, the term “plurality” can be 2, 3, 4, 5, 6, 7, 8, 9, 10, or more.

As used herein, the term “set of” means one or more. For example, a set of items includes one or more items.

As used herein, the phrase “at least one of,” when used with a list of items, means different combinations of one or more of the listed items may be used and only one of the items in the list may be needed. The item may be a particular object, thing, step, operation, process, or category. In other words, “at least one of” means any combination of items or number of items may be used from the list, but not all of the items in the list may be required. For example, without limitation, “at least one of item A, item B, or item C” means item A; item A and item B; item B; item A, item B, and item C; item B and item C; or item A and C. In some cases, “at least one of item A, item B, or item C” means, but is not limited to, two of item A, one of item B, and ten of item C; four of item B and seven of item C; or some other suitable combination.

As used herein, a “model” may include one or more algorithms, one or more mathematical techniques, one or more machine learning algorithms, or a combination thereof

As used herein, “machine learning” may be the practice of using algorithms to parse data, learn from it, and then make a determination or prediction about something in the world. Machine learning may use algorithms that can learn from data without relying on rules-based programming

As used herein, an “artificial neural network” or “neural network” (NN) may refer to mathematical algorithms or computational models that mimic an interconnected group of artificial neurons that processes information based on a connectionistic approach to computation. Neural networks, which may also be referred to as neural nets, may employ one or more layers of linear units, nonlinear units, or both to predict an output for a received input. Some neural networks include one or more hidden layers in addition to an output layer. The output of each hidden layer may be used as input to the next layer in the network, i.e., the next hidden layer or the output layer. Each layer of the network may generate an output from a received input in accordance with current values of a respective set of parameters. In the various embodiments, a reference to a “neural network” may be a reference to one or more neural networks.

A neural network may process information in two ways. For example, a neural network may process information when it is being trained in training mode and when it puts what it has learned into practice in inference (or prediction) mode. Neural networks may learn through a feedback process (e.g., backpropagation) which allows the network to adjust the weight factors (modifying its behavior) of the individual nodes in the intermediate hidden layers so that the output matches the outputs of the training data. In other words, a neural network may learn by being fed training data (learning examples) and eventually learns how to reach the correct output, even when it is presented with a new range or set of inputs. A neural network may include, for example, without limitation, at least one of a Feedforward Neural Network (FNN), a Recurrent Neural Network (RNN), a Modular Neural Network (MNN), a Convolutional Neural Network (CNN), a Residual Neural Network (ResNet), an Ordinary Differential Equations Neural Networks (neural-ODE), a Squeeze and Excitation embedded neural network, a MobileNet, or another type of neural network.

As used herein, “deep learning” may refer to the use of multi-layered artificial neural networks to automatically learn representations from input data such as images, video, text, etc., without human provided knowledge, to deliver highly accurate predictions in tasks such as object detection/identification, speech recognition, language translation, etc.

### III. Neovascular Age-Related Macular Degeneration (NAMD) Treatment Management

### III.A. Exemplary Treatment Management System

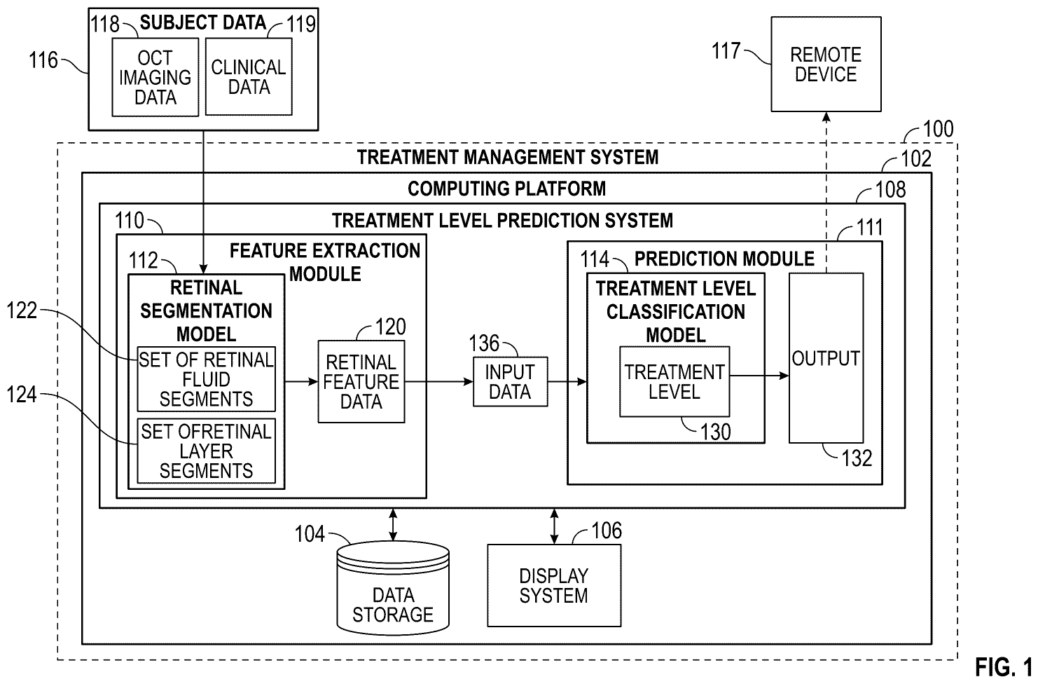

Referring now to the figures, FIG. 1 is a block diagram of a treatment management system 100 in accordance with one or more embodiments. Treatment management system 100 may be used to manage the treatment for a subject diagnosed with neovascular age-related macular degeneration (nAMD). In one or more embodiments, treatment management system 100 includes computing platform 102, data storage 104, and display system 106. Computing platform 102 may take various forms. In one or more embodiments, computing platform 102 includes a single computer (or computer system) or multiple computers in communication with each other. In other examples, computing platform 102 takes the form of a cloud computing platform, a mobile computing platform (e.g., a smartphone, a tablet, etc.), or a combination thereof.

Data storage 104 and display system 106 are each in communication with computing platform 102. In some examples, data storage 104, display system 106, or both may be considered part of or otherwise integrated with computing platform 102. Thus, in some examples, computing platform 102, data storage 104, and display system 106 may be separate components in communication with each other, but in other examples, some combination of these components may be integrated together.

### III.A.i. Prediction Mode

Treatment management system 100 includes treatment level prediction system 108, which may be implemented using hardware, software, firmware, or a combination thereof. In one or more embodiments, treatment level prediction system 108 is implemented in computing platform 102. Treatment level prediction system 108 includes feature extraction module 110 and prediction module 111. Each of feature extraction module 110 and prediction module 111 may be implemented using hardware, software, firmware, or a combination thereof.

In one or more embodiments, each of feature extraction module 110 and prediction module 111 is implemented using one or more machine learning models. For example, feature extraction module 110 may be implemented using a retinal segmentation model 112, while prediction module 111 may be implemented using a treatment level classification model 114.

Retinal segmentation model 112 is used at least to process OCT imaging data 118 and generate segmented images that identify one or more retinal pathologies (e.g., retinal fluids), one or more retinal layers, or both. In one or more embodiments, retinal segmentation model 112 takes the form of a machine learning model. For example, retinal segmentation model 112 may be implemented using a deep learning model. The deep learning model may be comprised of, for example, but is not limited to, one or more neural networks.

In one or more embodiments, treatment level classification model 114 may be used to classify a treatment level for the treatment. This classification may be, for example, a binary (e.g., high and low; or high and not high) classification. In other embodiments, some other type of classification may be used (e.g., high, moderate, and low). In one or more embodiments, treatment level classification model 114 is implemented using a symbolic model, which may be also referred to as a feature-based model. The symbolic model may include, for example, but is not limited to, an Extreme Gradient Boosting (XGBoost) algorithm.

Feature extraction module 110 receives subject data 116 for a subject diagnosed with nAMD as input. The subject may be, for example, a patient that is undergoing, has undergone, or will undergo treatment for the nAMD condition. Treatment may include, for example, an anti-vascular endothelial growth factor (anti-VEGF) agent, which may be administered via a number of injections (e.g., intravitreal injections).

Subject data 116 may be received from a remote device (e.g., remote device 117), retrieved from a database, or received in some other manner In one or more embodiments, subject data 116 is retrieved from data storage 104.

Subject data 116 includes optical coherence tomography (OCT) imaging data 118 of a retina of the subject diagnosed with nAMD. OCT imaging data 118 may include, for example, spectral domain optical coherence tomography (SD-OCT) imaging data. In one or more embodiments, OCT imaging data 118 includes one or more SD-OCT images captured at a time prior to treatment, a time just before treatment, a time just after a first treatment, another point in time, or a combination thereof. In some examples, OCT imaging data 118 includes one or more images generated during an initial phase (e.g., a 3-month initial phase for months M0-M2) of treatment. During the initial phase, treatment is administered monthly via injection over 3 months.

In one or more embodiments, subject data 116 further includes clinical data 119. Clinical data 119 may include, for example, data for a set of clinical features. The set of clinical features may include, for example, but is not limited to, best corrected visual acuity (BCVA) (e.g., for a baseline point in time prior to treatment), central subfield thickness (CST) (e.g., extracted from one or more OCT images), pulse, systolic blood pressure (SBP), diastolic blood pressure (DBP), or a combination thereof. This clinical data 119 may have been generated at a baseline point in time prior to treatment and/or at another point in time during a treatment phase.

Feature extraction module 110 uses OCT imaging data 118 to extract retinal feature data 120 for a plurality of retinal features. Retinal feature data 120 includes values for various features associated with the retina of a subject. For example, retinal feature data 120 may include values for various features associated with one or more retinal pathologies (e.g., retinal fluids), one or more retinal layers, or both. Examples of retinal fluids include, but are not limited to, an intraretinal fluid (IRF), a subretinal fluid (SRF), a fluid associated with pigment epithelial detachment (PED), and a subretinal hyperreflective material (SHRM). Examples of retinal layers include, but are not limited to, an internal limiting membrane (ILM) layer, an outer plexiform layer-Henle fiber layer (OPL-HFL), an inner boundary-retinal pigment epithelial detachment (IB-RPE), an outer boundary-retinal pigment epithelial detachment (OB-RPE), and a Bruch's membrane (BM).

In one or more embodiments, feature extraction module 110 inputs at least a portion of subject data 116 (e.g., OCT imaging data 118) into retinal segmentation model 112 (e.g., a deep learning model) to identify one or more retinal segments. For example, retinal segmentation model 112 may generate a segmented image (e.g., segmented OCT image) that identifies, by pixel, one or more retinal segments. A retinal segment may be, for example, an identification of a portion of the image as a retinal pathology (e.g., fluid), a boundary of a retina layer, or a retinal layer. For example, retinal segmentation model 112 may generate a segmented image that identifies set of retinal fluid segments 122, set of retinal layer segments 124, or both. Each segment of set of retinal fluid segments 122 corresponds to a retinal fluid. Each segment of set of retinal layers 124 corresponds to a retinal layer.

In one or more embodiments, retinal segmentation model 112 has been trained to output an image that identifies set of retinal fluid segments 122 and an image that identifies set of retinal layer segments 124. Feature extraction module 110 may then identify retinal feature data 120 using these images identifying set of retinal fluid segments 122 and set of retinal layer segments 124. For example, feature extraction module 110 may perform measurements, computations, or both using the images to identify retinal feature data 120. In other embodiments, retinal segmentation model 112 is trained to output retinal feature data 120 based on set of retinal fluid segments 122, set of retinal layer segments 124, or both.

Retinal feature data 120 may include, for example, one or more values identified (e.g., computed, measured, etc.) based on set of retinal fluid segments 122, the set of retinal layer segments 124, or both. For example, retinal feature data 120 may include a value for a corresponding retinal fluid segment of set of retinal fluid segments 122. This value may be for a volume, a height, a width, or some other measurement of the retinal fluid segment. In one or more embodiments, retinal feature data 120 includes a value for a corresponding retinal layer segment of the set of retinal layer segments 124. For example, the value may include a minimum thickness, a maximum thickness, an average thickness, or another measurement or computed value associated with the retinal layer segment. In some cases, retinal feature data 120 includes a value that is computed using more than one fluid segments of set of retinal fluid segments 122, more than one retinal layer segment of set of retinal layer segments 124, or both.

Feature extraction module 110 generates an output using retinal feature data 120, this output forms input data 126 for prediction module 111. Input data 126 may be formed in various ways. In one or more embodiments, the input data 126 includes the retinal feature data 120. In other embodiments, some portion or all of the retinal feature data 120 may be modified, combined, or integrated to form the input data 126. In some examples, two or more values in retinal feature data 120 may be used to compute a value that is included in input data 126. In one or more embodiments, input data 126 includes clinical data 119 for the set of clinical features.

Prediction module 111 uses input data 126 received from feature extraction module 110 to predict treatment level 130. Treatment level 130 may be a classification for the number of injections predicted to be needed for a subject. The number of injections needed for the subject may be based on, for example, without limitation, one or more The number of injections needed for the subject may be an overall number of injections or a number of injections within a selected period of time. For example, treatment of a subject may include an initial phase and a pro re nata (PRN) or as needed phase. Prediction module 111 may be used to predict treatment level 130 for the PRN phase. In some examples, the time period for the PRN phase includes the 21 months after the initial phase. In these examples, treatment level 130 is a classification of “high” or “low” with “high” being defined as 16 or more injections during the PRN phase and “low” being defined as 5 or fewer injections during the PRN phase.

As noted above, treatment level 130 may include a classification for the number of injections that is predicted for treatment of the subject during the PRN phase, a number of injections during the PRN phase or another time period, an injection frequency, another indicator of treatment requirements for the subject, or a combination thereof.

In one or more embodiments, prediction module 111 sends input data 126 into treatment level classification model 114 to predict treatment level 130. For example, treatment level classification model 114 (e.g., XGBoost algorithm) may have been trained to predict treatment level 130 based on input data 126.

In one or more embodiments, prediction module 111 generates output 132 using treatment level 130. In some examples, output 132 includes treatment level 130. In other examples, output 132 includes information generated based on treatment level 130. For example, when treatment level 130 identifies a number of injections predicted for treatment of the subject during the PRN phase, output 132 may include a classification for this treatment level. In another example, treatment level 130 that is predicted by treatment level classification model 114 includes a number of injections and a classification (e.g., high, low, etc.) for the number of injections, and output 132 includes only the classification. In another example, output 132 includes the name of the treatment, the dosage of the treatment, or both.

In one or more embodiments, output 132 may be sent to remote device 117 over one or more communication links (e.g., wired, wireless, and/or optical communications links). For example, remote device 117 may be a device or system such as a server, a cloud storage, a cloud computing platform, a mobile device (e.g., mobile phone, tablet, a smartwatch, etc.), some other type of remote device or system, or a combination thereof. In some embodiments, output 132 is transmitted as a report that may be viewed on remote device 138. The report may include, for example, without limitation, at least one of a table, a spreadsheet, a database, a file, a presentation, an alert, a graph, a chart, one or more graphics, or a combination thereof.

In one or more embodiments, output 132 may be displayed on display system 106, stored in data storage 104, or both. Display system 106 includes one or more display devices in communication with computing platform 102. Display system 106 may be separate from or at least partially integrated as part of computing platform 102.

Treatment level 130, output 132, or both may be used to manage the treatment of the subject diagnosed with nAMD. The prediction of treatment level 130 may enable, for example, a clinician

### III.A.ii Training Mode

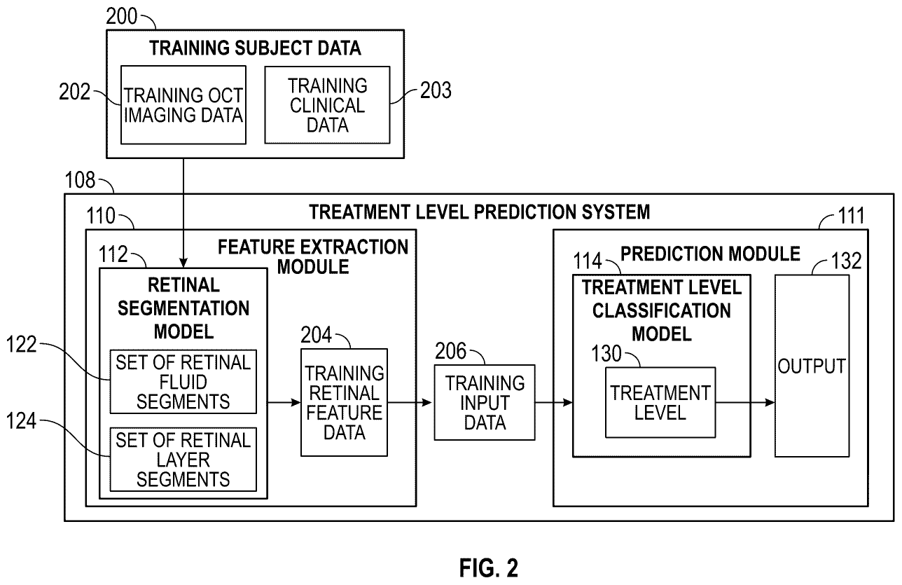

FIG. 2 is a block diagram of treatment level prediction system 108 from FIG. 1 being used in a training mode in accordance with one or more embodiments. In the training mode, retinal segmentation model 112 of feature extraction module 110 and treatment level classification model 114 of prediction module 111 are trained using training subject data 200. Training subject data 200 may include, for example, training OCT imaging data 202. In some embodiments, training subject data 200 includes training clinical data 203.

Training OCT imaging data 202 may include, for example, SD-OCT images capturing the retinas of subjects receiving anti-VEGF injections over an initial phase of treatment (e.g., first 3 months, first 5 months, first 9 months, first 10 months, etc.), a PRN phase of treatment (e.g., the 5 to 25 months following the initial phase), or both. In one or more embodiments, training OCT imaging data 202 includes a first portion of SD-OCT images for subjects who received injections of 0.5 mg of ranibizumab over a PRN phase of 21 months and a second portion of SD-OCT images for subjects who received injections of 2.0 mg of ranibizumab over a PRN phase of 21 months. In other embodiments, OCT images for subjects who received injections of other dosages (e.g., between 0.25 mg and 3 mg) of injections may be included, OCT images for subjects who were monitored over a longer or shorter PRN phase be included, OCT images for subjects who were given a different anti-VEGF agent may be included, or a combination thereof may be included.

Training clinical data 203 may include, for example, data for a set of clinical features for the training subjects. The set of clinical features may include, for example, but is not limited to, best corrected visual acuity (BCVA) (e.g., for a baseline point in time prior to treatment), central subfield thickness (CST) (e.g., extracted from one or more OCT images), pulse, systolic blood pressure (SBP), diastolic blood pressure (DBP), or a combination thereof. The training clinical data 203 may have been generated at a baseline point in time prior to treatment (e.g., prior to the initial phase) and/or at another point in time during a treatment phase (e.g., between the initial phase and the PRN phase).

In one or more embodiments, retinal segmentation model 112 may be trained using training subject data 200 to generate segmented images that identify set of retinal fluid segments 122, set of retinal layer segments 124, or both. Set of retinal fluid segments 122 and set of retinal layer segments 124 may be segmented for each image in training OCT imaging data 202. Feature extraction module 110 generates training retinal feature data 204 using set of retinal fluid segments 122, set of retinal layer segments 124, or both. In one or more embodiments, feature extraction module 110 generates training retinal feature data 204 based on the output of retinal segmentation model 112. In other embodiments, retinal segmentation model 112 of feature extraction module 110 is trained to generate training retinal feature data 204 based on set of retinal fluid segments 122, set of retinal layer segments 124, or both.

Feature extraction module 110 generates an output using training retinal feature data 204 that forms training input data 206 for inputting into prediction module 111. Training input data 206 may include training retinal feature data 204 or may be generated based on training retinal feature data 204. For example, training retinal feature data 204 may be a filtered to form training input data 204. In one or more embodiments, training retinal feature data 204 is filtered to remove feature data for any subjects where more than 10% of the features of interest is missing data. In some examples, training retinal feature data 204 is filtered to remove retinal feature data for any subjects where complete data is not present for the entirety of the initial phase, the entirety of the PRN phase, or the entirety of both the initiation and PRN phases. In some embodiments, training input data 206 further includes training clinical data 203 or at least a portion of training clinical data 203.

Prediction module 111 receives training input data 206 and treatment level classification model 114 may be trained to predict treatment level 130 using training input data 206. In one or more embodiments, treatment level classification model 114 may be trained to predict treatment level 130 and to predict output 132 based on treatment level 130.

In other embodiments, training of treatment level prediction system 108 may include only the training of prediction module 111 and thereby, only the training of treatment level classification model 114. For example, retinal segmentation model 112 of feature extraction module 1110 may be pretrained to perform segmentation and/or generate feature data. Accordingly, training input data 206 may be received from another source (e.g., data storage in FIG. 1, remote device 117 in FIG. 1, some other device, etc.).

### III.B. Exemplary Methodologies for Managing NAMD Treatment

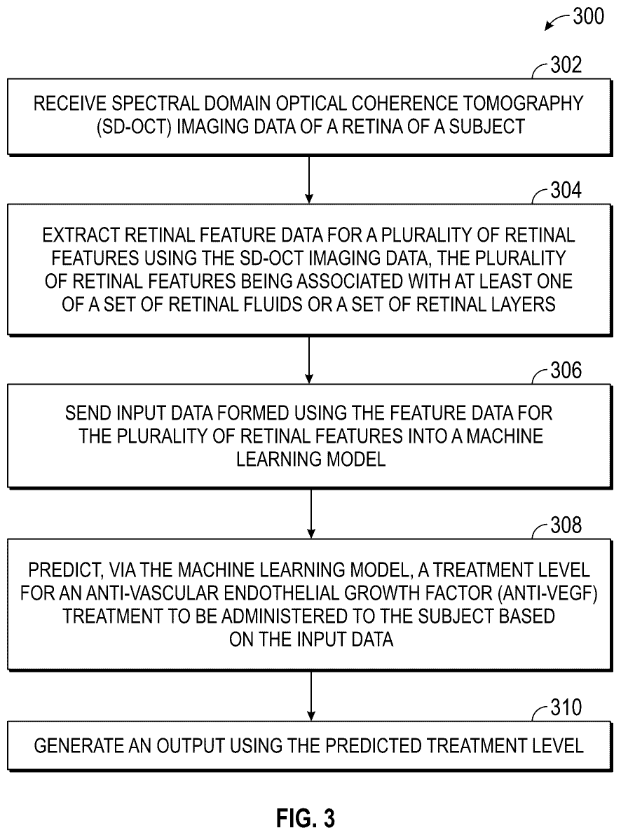

FIG. 3 is a flowchart of a process 300 for managing a treatment for a subject diagnosed with nAMD in accordance with one or more embodiments. In one or more embodiments, process 300 is implemented using treatment management system 100 described in FIG. 1. More specifically, process 300 may be implemented using treatment level prediction system 108 in FIG. 1. For example, process 300 may be used to predict a treatment level 130 based on subject data 116 (e.g., OCT imaging data 118) in FIG. 1.

Step 302 includes receiving spectral domain optical coherence tomography (SD-OCT) imaging data of a retina of a subject. In step 302, the SD-OCT imaging data may be one example of an implementation for OCT imaging data 118 in FIG. 1. In one or more embodiments, the SD-OCT imaging data may be received from a remote device, retrieved from a database, or received in some other manner The SD-OCT imaging data received in step 302 may include, for example, one or more SD-OCT images captured at a baseline point in time, a point in time just before treatment, a point in time just after treatment, another point in time, or a combination thereof. In one or more examples, the SD-OCT imaging data includes one or more images generated at a baseline point in time prior to any treatment (e.g., Day 0), at a point in time around a first month's injection (e.g., M1), at a point in time around a second month's injection (e.g., M2), at a point in time around a first third month's injection (e.g., M3), or a combination thereof.

Step 304 includes extracting retinal feature data for a plurality of retinal features using the SD-OCT imaging data, the plurality of retinal features being associated with at least one of a set of retinal fluids or a set of retinal layers. In one or more embodiments, step 304 may be implemented using the feature extraction module 110 in FIG. 1. For example, feature extraction model 110 may be used to extract retinal feature data 120 for a plurality of retinal features associated with at least one of set of retinal fluid segments 122 or set of retinal layer segments 124 using the SD-OCT imaging data received in step 302. In step 304, the retinal feature data may take the form of, for example, retinal feature data 120 in FIG. 1.

In some examples, the retinal feature data includes a value (e.g., computed value, measurement, etc.) that corresponds to one or more retinal fluids, one or more retinal layers, or both. Examples of retinal fluids include, but are not limited to, an intraretinal fluid (IRF), a subretinal fluid (SRF), a fluid associated with pigment epithelial detachment (PED), and a subretinal hyperreflective material (SHRM). A value for a feature associated with a corresponding retinal fluid my include, for example, a value for a volume, a height, or a width of the corresponding retinal fluid. Examples of retinal layers include, but are not limited to, an internal limiting membrane (ILM) layer, an outer plexiform layer-Henle fiber layer (OPL-HFL), an inner boundary-retinal pigment epithelial detachment (IB-RPE), an outer boundary-retinal pigment epithelial detachment (OB-RPE), and a Bruch's membrane (BM). A value for a feature associated with a corresponding retinal layer may include, for example, a value for a minimum thickness, a maximum thickness, or an average thickness of the corresponding retinal layer. In some cases, a retinal layer-associated feature may correspond to more than one retinal layer (e.g., a distance between the boundaries of two retinal layers).

In one or more embodiments, the plurality of retinal features in step 304 includes at least one feature associated with a subretinal fluid (SRF) of the retina and at least one feature associated with pigment epithelial detachment (PED).

In one or more embodiments, the SD-OCT imaging data includes an SD-OCT image captured during a single clinical visit. In some embodiments, the SD-OCT imaging data includes SD-OCT images captured at multiple clinical visits (e.g., at every month of an initial phase of treatment). In one or more embodiments, step 304 includes extracting the retinal feature data using the SD-OCT imaging data via a machine learning model (e.g., retinal segmentation model 112 in FIG. 1). The machine learning model may include, for example, a deep learning model. In one or more embodiments, the deep learning model includes one or more neural networks, each of which may be, for example, a convolutional neural network (CNN).

Step 306 includes sending input data formed using the retinal feature data for the plurality of retinal features into a machine learning model. In step 306, input data may take the form of, for example, input data 126 in FIG. 1. In some embodiments, the input data includes the retinal feature data extracted in step 304. In other words, the retinal feature data or at least a portion of the retinal feature data may be sent on as the input data for the machine learning model. In other embodiments, some portion or all of the retinal feature data may be modified, combined, or integrated to form the input data. The machine learning model in step 306 may be, for example, treatment level classification model 114 in FIG. 1. In one or more embodiments, the machine learning model may be a symbolic model (feature-based model) (e.g., model using the XGBoost algorithm).

In some embodiments, the input data may further include clinical data for a set of clinical features for the subject. The clinical data may be, for example, clinical data 117 in FIG. 1. The set of clinical features may include, for example, but is not limited to, best corrected visual acuity (BCVA) (e.g., for a baseline point in time prior to treatment), central subfield thickness (CST) (e.g., extracted from one or more OCT images), pulse, systolic blood pressure (SBP), diastolic blood pressure (DBP), or a combination thereof. The input data may include all or some of the retinal feature data described above.

Step 308 includes predicting, via the machine learning model, a treatment level for an anti-vascular endothelial growth factor (anti-VEGF) treatment to be administered to the subject based on the input data. The treatment level may include a classification for the number of injections that is predicted for the anti-VEGF treatment of the subject (e.g., during the PRN phase of treatment), a number of injections (e.g., during the PRN phase or another time period), an injection frequency, another indicator of treatment requirements for the subject, or a combination thereof.

Process 300 may optionally include step 310. Step 310 includes generating an output using the predicted treatment level. The output may include the treatment level and/or information generated based on the predicted treatment level. In some embodiments, step 310 further includes sending the output to a remote device. The output may be, for example, a report that can be used to guide a clinician, the subject, or both with respect to the subject's treatment. For example, if the predicted treatment level indicates that the subject may need a “high” level of injections over a PRN phase, the output may identify certain protocols that can be put in place to help ensure subject compliance (e.g., the subject showing up to injection appointments, evaluation appointments).

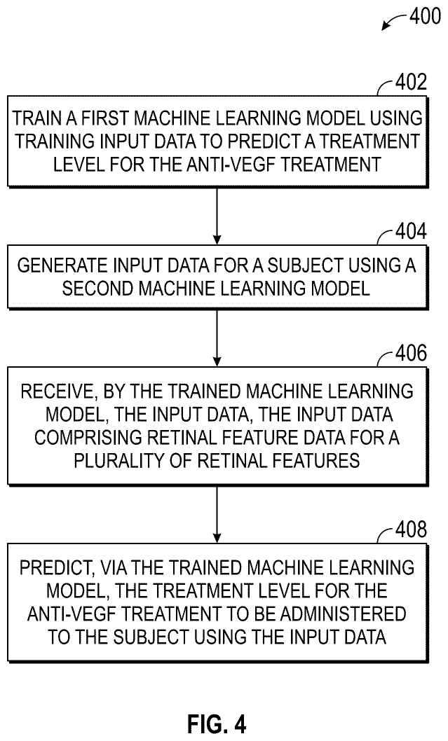

FIG. 4 is a flowchart of a process 400 for managing a treatment for a subject diagnosed with nAMD in accordance with one or more embodiments. In one or more embodiments, process 400 is implemented using the treatment management system 100 described in FIG. 1. More specifically, process 400 may be implemented using treatment level prediction system 108 in FIGS. 1 and 2.

Step 402 includes training a first machine learning model using training input data to predict a treatment level for the anti-VEGF treatment. The training input data may be, for example, training input data 206 in FIG. 2. The training input data may be formed using training OCT imaging data such as, for example, training OCT imaging data 202 in FIG. 2. The first machine learning model may include, for example, a symbolic model such as an XGBoost model.

In one or more embodiments, the training OCT imaging data is automatically segmented using a second machine learning model to generate segmented images (segmented OCT images). The second machine learning model may include, for example, a deep learning model. Retinal feature data is extracted from the segmented images and used to form the training input data. For example, at least a portion of the retinal feature data is used to form at least a portion of the training input data. In some examples, the training input data may further include training clinical data (e.g., measurements for BCVA, pulse, systolic blood pressure, diastolic blood pressure, CST, etc.).

The training input data my include data for a first portion of training subject treated with a first dosage (e.g., 0.5 mg) of the anti-VEGF treatment and data for a second portion of training subject treated with a second dosage (e.g., 2.0 mg) of the anti-VEGF treatment. The training input data may be data corresponding to a pro re nata phase of treatment (e.g., 21 months after an initial phase of treatment that includes monthly injections, 9 months after an initial phase of treatment, or some other period of time).

In one or more embodiments, the retinal feature data may be preprocessed to form the training input data. For example, the values for retinal features corresponding to multiple visits (e.g., 3 visits) may be concatenated. In some examples, highly correlated features may be excluded from the training input data. For example, in step 402, clusters of highly correlated (e.g., correlation coefficient above 0.9) features may be identified. For each pair of highly correlated features, the value for one of these features may be randomly selected for exclusion from the training input data. For clusters of 3 or more highly correlated features, the values for those features that are the correlated with the most other features in the cluster are iteratively excluded (e.g., until a single feature of the cluster remains). These examples of preprocessing may be only one example of the types of preprocessing that may be performed on the retinal feature data.

In still other embodiments, step 402 includes training the first machine learning model with respect to a first plurality of retinal features. Feature importance analysis may be used to determine which of the first plurality of retinal features are most important to predicting treatment level. In these embodiments, step 402 may include reducing the first plurality of retinal features to a second plurality of retinal features (e.g., 3, 4, 5, 6, 7, . . . 10, or some other number of retinal features). The first machine learning model may then be trained to use the second plurality of retinal features in predicting treatment level.

Step 404 includes generating input data for a subject using the second machine learning model. The input data for the subject may be generated using retinal feature data extracted from OCT imaging data of a retina of the subject using the second machine learning model, clinical data, or both. For example, the second machine learning model may be a pretrained to identify a set of retinal fluid segments, a set of retinal layer segments, or both in OCT images. The set of retinal fluid segments, the of retinal layer segments, or both may then be used to identify the retinal feature data for a plurality of retinal features via computation, measurement, etc. In some embodiments, the second machine learning model may be pretrained to identify the retinal feature data based on the set of retinal fluid segments, the set of retinal layer segments, or both .

Step 406 includes receiving, by the trained machine learning model, the input data, the input data comprising retinal feature data for a plurality of retinal features. The input data may additionally include clinical data for a set of clinical features.

Step 408 includes predicting, via the trained machine learning model, the treatment level for the anti-VEGF treatment to be administered to the subject using the input data. The treatment level may be, for example, a classification of “high” or “low” (or “high” and “not high”). A level of “high” may indicate, for example, 10, 11, 12, 13, 14, 15, 16, 17, or 18 more injections during a PRN phase (e.g., a time period of 8, 9, 10, 11, 12, 13, 14, 15, 16, 17, 18, 10, 20, 21, 22, 23, 24, or some other number of months). A level of “low” may indicate, for example, 7, 6, 5, 4, or fewer injections during the PRN phase.

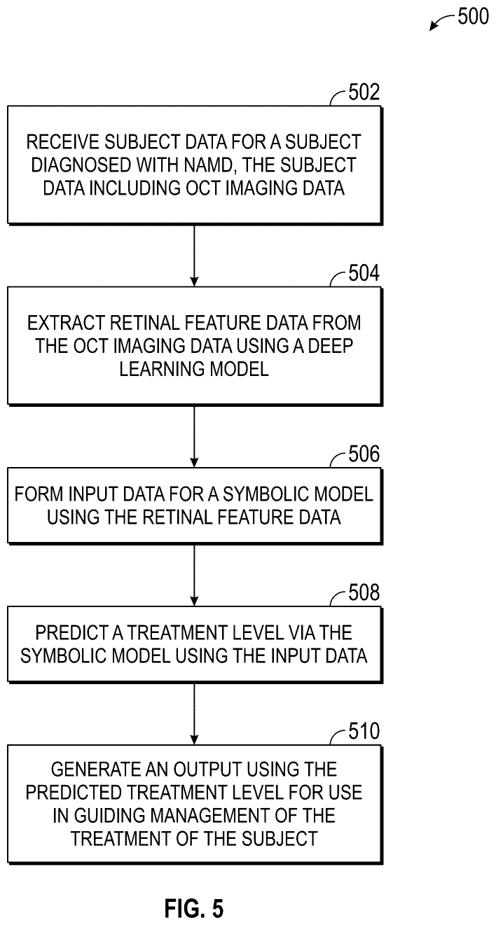

FIG. 5 is a flowchart of a process 500 for managing a treatment for a subject diagnosed with nAMD in accordance with one or more embodiments. This process 500 may be implemented using, for example, treatment management system 100 in FIG. 1.

Step 502 may include receiving subject data for a subject diagnosed with nAMD, the subject data including OCT imaging data. The OCT imaging data may be, for example, SD-OCT imaging data. The OCT imaging data may include one or more OCT (e.g., SD-OCT) images of the retina of the subject. In one or more embodiments, the subject data further includes clinical data. The clinical data may include, for example, a BCVA measurement (e.g., taken at a baseline point in time) and vitals (e.g., pulse, systolic blood pressure, diastolic blood pressure, etc.). In some embodiments, the clinical data includes central subfield thickness (CST) which may be a measurement extracted from one or more OCT images.

Step 504 includes extracting retinal feature data from the OCT imaging data using a deep learning model. In one or more embodiments, the deep learning model is used to segment out a set of fluid segments and a set of retinal layer segments from the OCT imaging data. For example, the deep learning model may be used to set segment out a set of fluid segments and a set of retinal layer segments from each OCT image of the OCT imaging data to produce segmented images. These segmented images may be used to measure and/or compute values for a plurality of retinal features to form the retinal feature data. In other embodiments, the deep learning model may be used both perform the segmentation and generate the retinal feature data.

Step 506 includes forming input data for a symbolic model using the retinal feature data. The input data may include, for example, the retinal feature data. In other embodiments, the input data may be formed by modifying, integrating, or combining at least a portion of the retinal feature data to form new values. In still other embodiments, the input data may further include the clinical data described above.

Step 508 includes predicting a treatment level via the symbolic model using the input data. In one or more embodiments, the treatment level may be a classification of “high” or “low” (or “high” and “not high”). A level of “high” may indicate, for example, 10, 11, 12, 13, 14, 15, 16, 17, or 18 more injections during a PRN phase (e.g., a time period of 8, 9, 10, 11, 12, 13, 14, 15, 16, 17, 18, 10, 20, 21, 22, 23, 24, or some other number of months). A level of “low” may indicate, for example, 7, 6, 5, 4, or fewer injections during the PRN phase. A level of “not high” may indicate a number of injections below that required for the “high” classification.

Process 500 may optionally include step 510. Step 510 includes generating an output using the predicted treatment level for use in guiding management of the treatment of the subject. For example, the output may be a report, alert, notification, or other type of output that includes the treatment level. In some examples, the output includes a set of protocols based on the predicted treatment level. For example, if the predicted treatment level is “high,” the output may outline a set of protocols that can be used to ensure subject compliance with evaluation appointments, injection appointments, etc. In some embodiments, the output may include certain information when the predicted treatment level is “high,” such as particular instructions for the subject or the clinician treating the subject, with this information being excluded from the output if the predicted treatment level is “low” or “not high.” Thus, the output may take various forms depending on the predicted treatment level.

### III. C. Exemplary Segmented Images

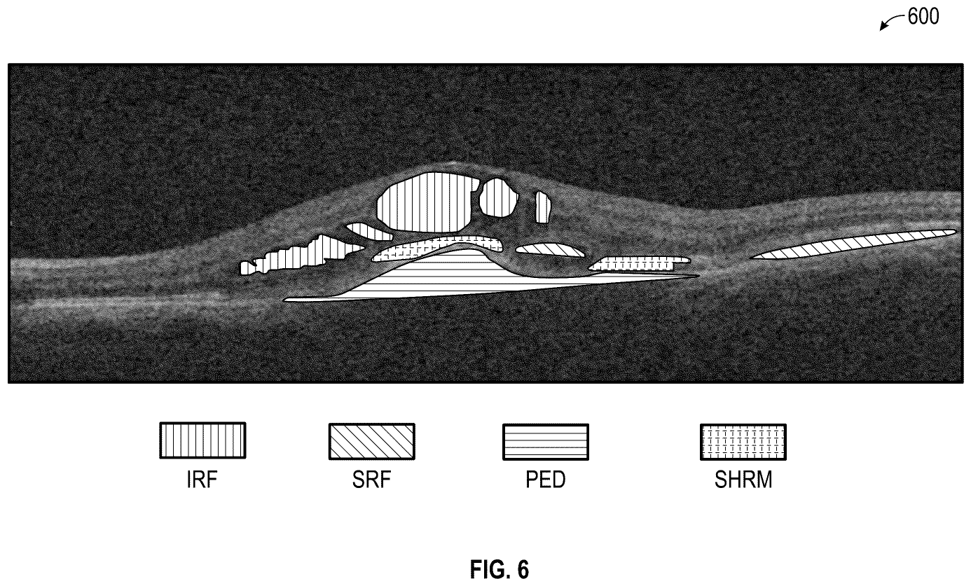

FIG. 6 is an illustration of a segmented OCT image in accordance with one or more embodiments. Segmented OCT image 600 may have been generated using, for example, retinal segmentation model 112 in FIG. 1. Segmented OCT image 600 identifies set of retinal fluid segments 602, which may be one example of an implementation for set of retinal fluid segments 122 in FIG. 1. Set of retinal fluid segments 602 identify an intraretinal fluid (IRF), a subretinal fluid (SRF), a fluid associated with pigment epithelial detachment (PED), and subretinal hyperreflective material (SHRM).

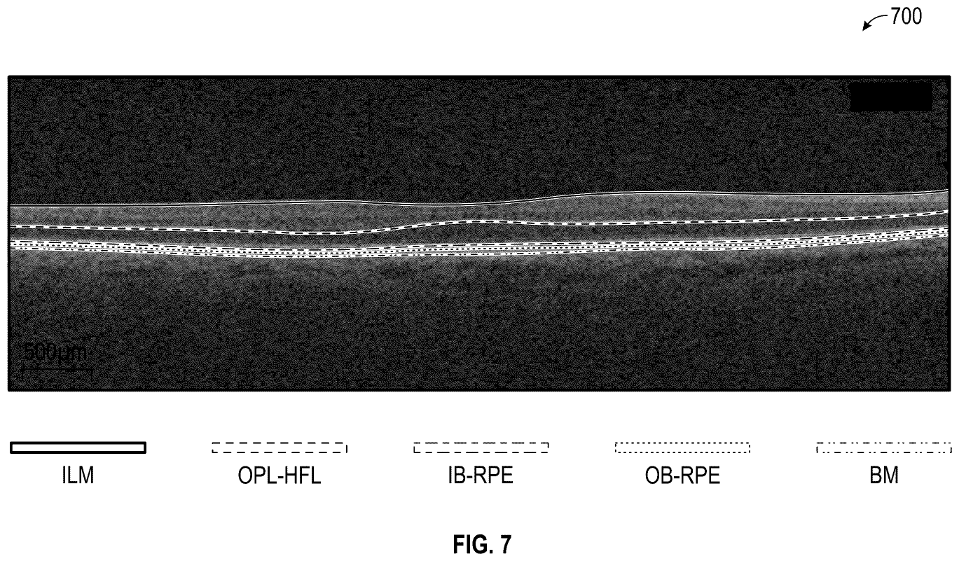

FIG. 7 is an illustration of a segmented OCT image in accordance with one or more embodiments. Segmented OCT image 700 may have been generated using, for example, retinal segmentation model 112 in FIG. 1. Segmented OCT image 700 identifies set of retinal layer segments 702, which may be one example of an implementation for set of retinal layer segments 124 in FIG. 1. Set of retinal layer segments 702 identify an internal limiting membrane (ILM) layer, an outer plexiform layer-Henle fiber layer (OPL-HFL), an inner boundary-retinal pigment epithelial detachment (IB-RPE), an outer boundary-retinal pigment epithelial detachment (OB-RPE), and a Bruch's membrane (BM).

### IV. Exemplary Experimental Data

### IV.A. Study #1

In a first study, a machine learning model (e.g., symbolic model) was trained using training input data generated from training OCT imaging data. For example, SD-OCT imaging data for 363 training subjects of the HARBOR clinical trial (NCT00891735) from two different ranibizumab PRN arms (one with 0.5 mg dosing, one with 2.0 mg dosing) were collected. The SD-OCT imaging data included monthly SD-OCT images, where applicable, for a 3-month initial phase of treatment and a 21-month PRN phase of treatment. A “low” treatment level was classified as 5 or fewer injections during the PRN phase. A “high” treatment level was classified as 16 or more injections during the PRN phase.

A deep learning model was used to generate segmented images for each month of the initial phase (e.g., identifying a set of fluid segments and a set of retinal layer segments in each SD-OCT image). Accordingly, 3 fluid-segmented images and 3 layer-segmented images were generated (one for each visit). Training retinal feature data was computed for each training subject case using these segmented images. The training retinal feature data included data for 60 features computed using the fluid-segmented images and 45 features computed using the layer-segmented images. The training retinal feature data was computed for each of the three months of the initial phase. The training retinal feature data was combined with BCVA and CST data for each of the three months of the initial phase to form training input data. The training input data was filtered to remove any subject cases where data for more than 10% of the 105 total retinal features was missing and to remove any subject cases where complete data was not available for the full 24 months of both the initial phase and the PRN phase.

The filtered training input data was then input into a symbolic model implemented using an XGBoost algorithm and evaluated using 5-fold cross validation. The symbolic model was trained using the training input data to classify a given subject as being associated with a “low” or “high” treatment level.

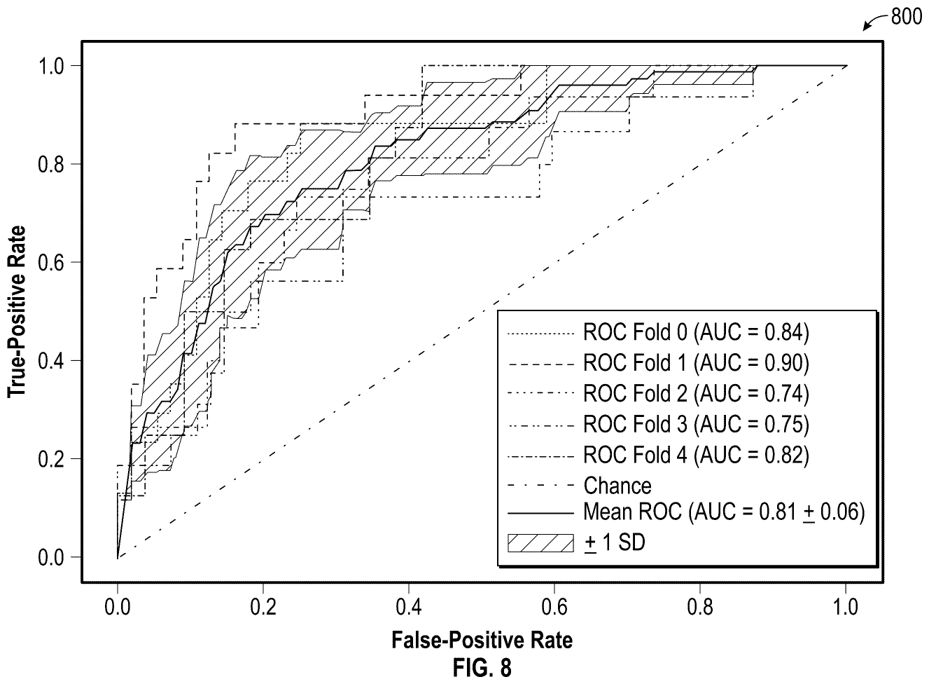

FIG. 8 is a plot illustrating the results of a 5-fold cross-validation for a treatment level classification of “low” in accordance with one or more embodiments. In particular, plot 800 provides validation data for the above-described experiment for subject cases classified with a “low” treatment level. The mean AUC for the “low” treatment level was 0.81±0.06.

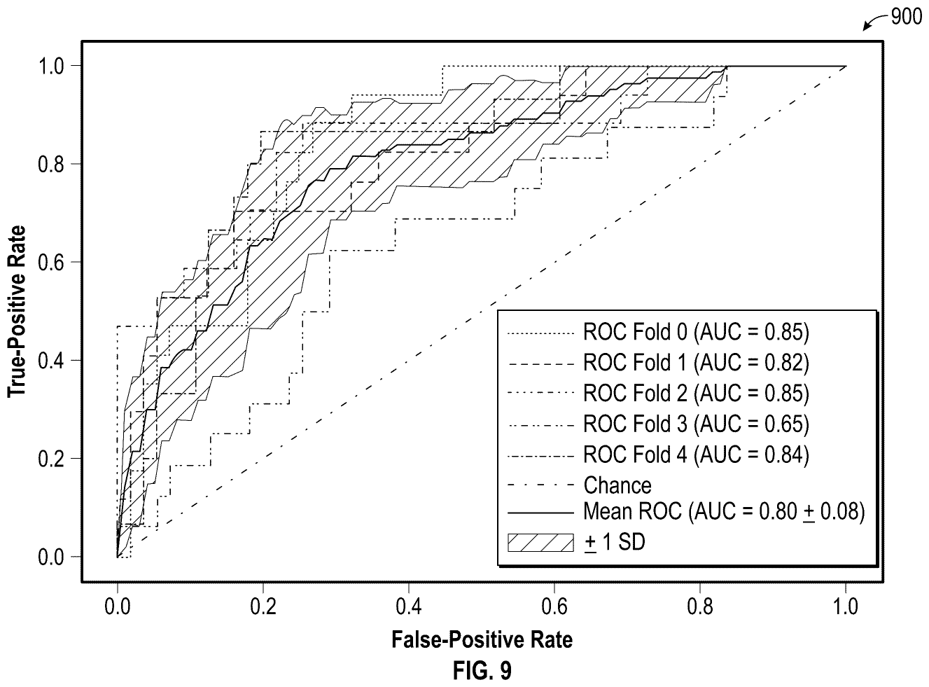

FIG. 9 is a plot illustrating the results of a 5-fold cross-validation for a treatment level classification of “high” in accordance with one or more embodiments. In particular, plot 900 provides validation data for the above-described experiment for subject cases classified with a “high” treatment level. The mean AUC for the “high” treatment level was 0.80±0.08.

The plot 800 in FIG. 8 and plot 900 in FIG. 9 show the feasibility of using a machine learning model (e.g., symbolic model) to predict low or high treatment levels for subjects with nAMD using retinal feature data extracted from automatically segmented SD-OCT images, the segmented SD-OCT images being generated using another machine learning model (e.g., deep learning model).

SHAP (Shapley Additive exPlanations) analysis was performed to determine the features most relevant to a treatment level classification of “low” and to the treatment level classification of “high.” For the treatment level classification of “low,” the 6 most important features included 4 features associated with retinal fluids (e.g., PED and SHRM), 1 feature associated with a retinal layer, and CST, with 5 of these 6 features being from month 2 of the initial phase of the treatment. The treatment level classification of “low” was most strongly associated with low volumes of detected PED height at month 2. For the treatment level classification of “high,” the 6 most important features included 4 features associated with retinal fluids (e.g., IRF and SHRM) and 2 features associated with retinal layers, with 4 of these 6 features being from month 2 of the initial phase of the treatment. The treatment level classification of “high” was most strongly associated with low volumes of detected SHRM at month 1.

### IV.B. Study #2

In a second study, a machine learning model (e.g., symbolic model) was trained using training input data generated from training OCT imaging data. For example, SD-OCT imaging data for 547 training subjects of the HARBOR clinical trial (NCT00891735) from two different ranibizumab PRN arms (one with 0.5 mg dosing, one with 2.0 mg dosing) were collected. The SD-OCT imaging data included monthly SD-OCT images, where applicable, for a 9-month initial phase of treatment and a 9-month PRN phase of treatment. Of the 547 training subjects, 144 were identified as having a “high” treatment level, which was classified as 6 or more injections during the PRN phase (9 visits between months 9 and 17).

A deep learning model was used to generate fluid-segmented and layer-segmented images from the SD-OCT imaging data collected at the visits at month 9 and month 10. Training retinal feature data was computed for each training subject case using these segmented images. For each of the visits at month 9 and month 10, the training retinal feature data included 69 features for retinal layers and 36 features for the retinal fluids.

This training retinal feature data was filtered to remove any subject cases where data for more than 10% of the retinal features was missing (e.g., failed segmentation) and to remove any subject cases where complete data was not available for the full 9 months of between month 9 and month 17 to thereby form input data.

This input data was input into a symbolic model for binary classification using the XGBoost algorithm with 5-fold cross-validation being repeated 10 times. The study was conducted was run for each feature group (the retinal fluid-associated features and the retinal layer-associated features) and on the combined set of all retinal features. Further, the study was conducted using features from only month 9 and from both month 9 and 10 together.

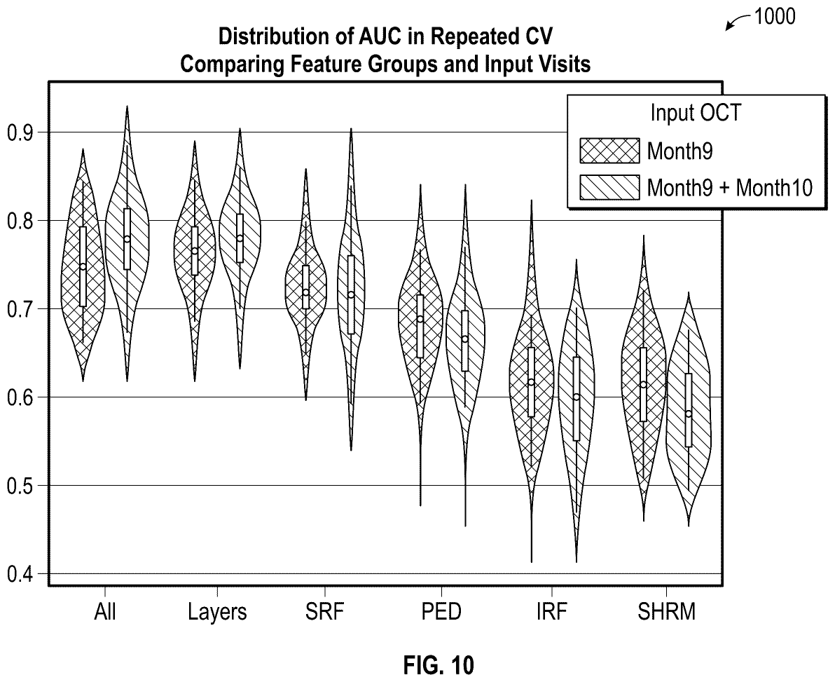

FIG. 10 is a plot of AUC data illustrating the results of repeated 5-fold cross-validation for a treatment level classification of “high” in accordance with one or more embodiments. As depicted in plot 1000, the best performance was achieved when using the features from all retinal layers. The AUC for using solely retinal layer-associated features was 0.76±0.04 when using month 9 data only and 0.79±0.05 when using month 9 and month 10 data together. These AUCs are close to the performance observed when using both retinal-layer associated features and retinal fluid-associated features. As depicted in plot 1000, adding the data from month 10 slightly improved performance. SHAP analysis confirmed that features associated with SRF and PED were among the most important features to predicting treatment level.

Thus, this study showed the feasibility of identifying future high treatment levels (e.g., 6 or more injections within a 9-month period that follows a 9-month period of initial treatment) for previously treated nAMD subjects using retinal featured data extracted from automatically segmented SD-OCT images.

### V. Computer-Implemented System

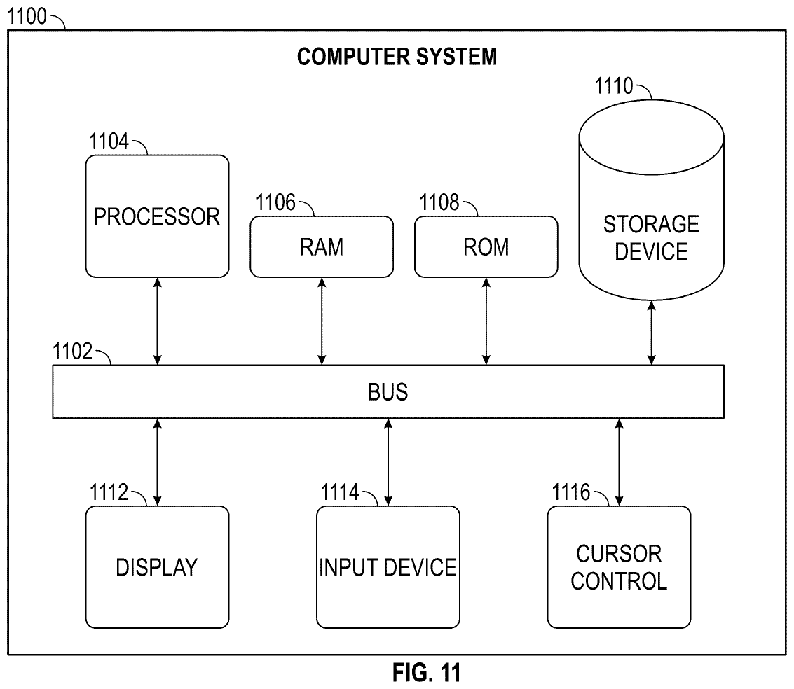

FIG. 11 is a block diagram illustrating an example of a computer system in accordance with one or more embodiments. Computer system 1100 may be an example of one implementation for computing platform 102 described above in FIG. 1. In one or more examples, computer system 1100 can include a bus 1102 or other communication mechanism for communicating information, and a processor 1104 coupled with bus 1102 for processing information. In various embodiments, computer system 1100 can also include a memory, which can be a random-access memory (RAM) 1106 or other dynamic storage device, coupled to bus 1102 for determining instructions to be executed by processor 1104. Memory also can be used for storing temporary variables or other intermediate information during execution of instructions to be executed by processor 1104. In various embodiments, computer system 1100 can further include a read-only memory (ROM) 1108 or other static storage device coupled to bus 1102 for storing static information and instructions for processor 1104. A storage device 1110, such as a magnetic disk or optical disk, can be provided and coupled to bus 1102 for storing information and instructions.

In various embodiments, computer system 1100 can be coupled via bus 1102 to a display 1112, such as a cathode ray tube (CRT) or liquid crystal display (LCD), for displaying information to a computer user. An input device 1114, including alphanumeric and other keys, can be coupled to bus 1102 for communicating information and command selections to processor 1104. Another type of user input device is a cursor control 1116, such as a mouse, a joystick, a trackball, a gesture-input device, a gaze-based input device, or cursor direction keys for communicating direction information and command selections to processor 1104 and for controlling cursor movement on display 1112. This input device 1114 typically has two degrees of freedom in two axes, a first axis (e.g., x) and a second axis (e.g., y), that allows the device to specify positions in a plane. However, it should be understood that input devices 1114 allowing for three-dimensional (e.g., x, y and z) cursor movement are also contemplated herein.

Consistent with certain implementations of the present teachings, results can be provided by computer system 1100 in response to processor 1104 executing one or more sequences of one or more instructions contained in RAM 1106. Such instructions can be read into RAM 1106 from another computer-readable medium or computer-readable storage medium, such as storage device 1110. Execution of the sequences of instructions contained in RAM 1106 can cause processor 1104 to perform the processes described herein. Alternatively, hard-wired circuitry can be used in place of or in combination with software instructions to implement the present teachings. Thus, implementations of the present teachings are not limited to any specific combination of hardware circuitry and software.

The term “computer-readable medium” (e.g., data store, data storage, storage device, data storage device, etc.) or “computer-readable storage medium” as used herein refers to any media that participates in providing instructions to processor 1104 for execution. Such a medium can take many forms, including but not limited to, non-volatile media, volatile media, and transmission media. Examples of non-volatile media can include, but are not limited to, optical, solid state, magnetic disks, such as storage device 1110. Examples of volatile media can include, but are not limited to, dynamic memory, such as RAM 1106. Examples of transmission media can include, but are not limited to, coaxial cables, copper wire, and fiber optics, including the wires that comprise bus 1102.

Common forms of computer-readable media include, for example, a floppy disk, a flexible disk, hard disk, magnetic tape, or any other magnetic medium, a CD-ROM, any other optical medium, punch cards, paper tape, any other physical medium with patterns of holes, a RAM, PROM, and EPROM, a FLASH-EPROM, any other memory chip or cartridge, or any other tangible medium from which a computer can read.

In addition to computer readable medium, instructions or data can be provided as signals on transmission media included in a communications apparatus or system to provide sequences of one or more instructions to processor 1104 of computer system 1100 for execution. For example, a communication apparatus may include a transceiver having signals indicative of instructions and data. The instructions and data are configured to cause one or more processors to implement the functions outlined in the disclosure herein. Representative examples of data communications transmission connections can include, but are not limited to, telephone modem connections, wide area networks (WAN), local area networks (LAN), infrared data connections, NFC connections, optical communications connections, etc.

It should be appreciated that the methodologies described herein, flow charts, diagrams, and accompanying disclosure can be implemented using computer system 1100 as a standalone device or on a distributed network of shared computer processing resources such as a cloud computing network.

The methodologies described herein may be implemented by various means depending upon the application. For example, these methodologies may be implemented in hardware, firmware, software, or any combination thereof. For a hardware implementation, the processing unit may be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, electronic devices, other electronic units designed to perform the functions described herein, or a combination thereof.

In various embodiments, the methods of the present teachings may be implemented as firmware and/or a software program and applications written in conventional programming languages such as C, C++, Python, etc. If implemented as firmware and/or software, the embodiments described herein can be implemented on a non-transitory computer-readable medium in which a program is stored for causing a computer to perform the methods described above. It should be understood that the various engines described herein can be provided on a computer system, such as computer system 1100, whereby processor 1104 would execute the analyses and determinations provided by these engines, subject to instructions provided by any one of, or a combination of, the memory components RAM 1106, ROM, 1108, or storage device 1110 and user input provided via input device 1114.

### VI. Recitation of Embodiments

Embodiment 1. A method for managing a treatment for a subject diagnosed with neovascular age-related macular degeneration (nAMD), the method comprising: receiving spectral domain optical coherence tomography (SD-OCT) imaging data of a retina of the subject; extracting retinal feature data for a plurality of retinal features using the SD-OCT imaging data, the plurality of retinal features being associated with at least one of a set of retinal fluids or a set of retinal layers; sending input data formed using the retinal feature data for the plurality of retinal features into a first machine learning model; and predicting, via the first machine learning model, a treatment level for an anti-vascular endothelial growth factor (anti-VEGF) treatment to be administered to the subject based on the input data.

Embodiment 2. The method of embodiment 1, wherein the retinal feature data includes a value associated with a corresponding retinal fluid of the set of retinal fluids, the value selected from a group consisting of a volume, a height, and a width of the corresponding retinal fluid.

Embodiment 3. The method of embodiment 1 or 2, wherein the retinal feature data includes a value for a corresponding retinal layer of the set of retinal layers, the value selected from a group consisting of a minimum thickness, a maximum thickness, and an average thickness of the corresponding retinal layer.

Embodiment 4. The method of any one of embodiments 1-3, wherein a retinal fluid of the set of retinal fluids is selected from a group consisting of an intraretinal fluid (IRF), a subretinal fluid (SRF), a fluid associated with pigment epithelial detachment (PED), or a subretinal hyperreflective material (SHRM).

Embodiment 5. The method of any one of embodiments 1-4, wherein a retinal layer of the set of retinal layers is selected from a group consisting of an internal limiting membrane (ILM) layer, an outer plexiform layer-Henle fiber layer (OPL-HFL), an inner boundary-retinal pigment epithelial detachment (IB-RPE), an outer boundary-retinal pigment epithelial detachment (OB-RPE), or a Bruch's membrane (BM).

Embodiment 6. The method of any one of embodiments 1-5, further comprising: forming the input data using the retinal feature data for the plurality of retinal features and clinical data for a set of clinical features, the set of clinical features including at least one of a best corrected visual acuity, a pulse, a diastolic blood pressure, or a systolic blood pressure.

Embodiment 7. The method of any one of embodiments 1-6, wherein predicting the treatment level comprises predicting a classification for the treatment level as either a high or a low treatment level.

Embodiment 8. The method of embodiment 7, wherein the high treatment level indicates sixteen or more injections of the anti-VEGF treatment during a selected time period after an initial phase of treatment.

Embodiment 9. The method of embodiment 7, wherein the low treatment level indicates five or fewer injections of the anti-VEGF treatment during a selected time period after an initial phase of treatment.

Embodiment 10. The method of any one of embodiments 1-9, wherein the extracting comprises: extracting the retinal feature data for the plurality of retinal features from segmented images generated using a second machine learning model that automatically segments the SD-OCT imaging data, wherein the plurality of retinal features is associated with at least one of a set of retinal fluid segments or a set of retinal layer segments identified in the segmented images.

Embodiment 11. The method of embodiment 10, wherein the second machine learning model comprises a deep learning model.

Embodiment 12. The method of any one of embodiments 1-11, wherein the first machine learning model comprises an Extreme Gradient Boosting (XGBoost) algorithm.

Embodiment 13. The method of any one of embodiments 1-12, wherein the plurality of retinal features includes at least one feature associated with subretinal fluid (SRF) and at least one feature associated with pigment epithelial detachment (PED).

Embodiment 14. The method of any one of embodiments 1-13, wherein the SD-OCT imaging data comprises an SD-OCT image captured during a single clinical visit.

Embodiment 15. A method for managing an anti-vascular endothelial growth factor (anti-VEGF) treatment for a subject diagnosed with neovascular age-related macular degeneration (nAMD), the method comprising: training a machine learning model using training input data to predict a treatment level for the anti-VEGF treatment, wherein the training input data is formed using training optical coherence tomography (OCT) imaging data; receiving input data for the trained machine learning model, the input data comprising retinal feature data for a plurality of retinal features; and predicting, via the trained machine learning model, the treatment level for the anti-VEGF treatment to be administered to the subject using the input data.

Embodiment 16. The method of embodiment 15, further comprising: generating the input data using the training OCT imaging data and a deep learning model, wherein the deep learning model is used to automatically segment the training OCT imaging data to form segmented images and wherein the retinal feature data is extracted from the segmented images.

Embodiment 17. The method of embodiment 15 or 16, wherein the machine learning model is trained to predict a classification for the treatment level as either a high treatment level or a low treatment level, wherein the high treatment level indicates sixteen or more injections of the anti-VEGF treatment during a selected time period after an initial phase of treatment.

Embodiment 18. The method of embodiment 15 or 16, wherein the machine learning model is trained to predict a classification for the treatment level as either a high treatment level or a not high treatment level, wherein the high treatment level indicates six or more injections of the anti-VEGF treatment during a selected time period after an initial phase of treatment.

Embodiment 19. A system for managing an anti-vascular endothelial growth factor (anti-VEGF) treatment for a subject diagnosed with neovascular age-related macular degeneration (nAMD), the system comprising: a memory containing machine readable medium comprising machine executable code; and a processor coupled to the memory, the processor configured to execute the machine executable code to cause the processor to:
receive spectral domain optical coherence tomography (SD-OCT) imaging data of a retina of the subject;
extract retinal feature data for a plurality of retinal features using the SD-OCT imaging data, the plurality of retinal features being associated with at least one of a set of retinal fluids or a set of retinal layers;
send input data formed using the retinal feature data for the plurality of retinal features into a first machine learning model; and
predict, via the first machine learning model, a treatment level for an anti-vascular endothelial growth factor (anti-VEGF) treatment to be administered to the subject based on the input data.

Embodiment 20. The system of embodiment 19, wherein the machine executable code further causes the processor to extract the retinal feature data for the plurality of retinal features from segmented images generated using a second machine learning model that automatically segments the SD-OCT imaging data, wherein the plurality of retinal features is associated with at least one of a set of retinal fluid segments or a set of retinal layer segments identified in the segmented images.

### VII. Additional Considerations

The headers and subheaders between sections and subsections of this document are included solely for the purpose of improving readability and do not imply that features cannot be combined across sections and subsection. Accordingly, sections and subsections do not describe separate embodiments.

Some embodiments of the present disclosure include a system including one or more data processors. In some embodiments, the system includes a non-transitory computer readable storage medium containing instructions which, when executed on the one or more data processors, cause the one or more data processors to perform part or all of one or more methods and/or part or all of one or more processes disclosed herein. Some embodiments of the present disclosure include a computer-program product tangibly embodied in a non-transitory machine-readable storage medium, including instructions configured to cause one or more data processors to perform part or all of one or more methods and/or part or all of one or more processes disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed Thus, it should be understood that although the present invention as claimed has been specifically disclosed by embodiments and optional features, modification and variation of the concepts herein disclosed may be resorted to by those skilled in the art, and that such modifications and variations are considered to be within the scope of this invention as defined by the appended claims.

The description provides preferred exemplary embodiments only, and is not intended to limit the scope, applicability or configuration of the disclosure. Rather, the description of the exemplary embodiments will provide those skilled in the art with an enabling description for implementing various embodiments. It is understood that various changes may be made in the function and arrangement of elements (e.g., elements in block or schematic diagrams, elements in flow diagrams, etc.) without departing from the spirit and scope as set forth in the appended claims.

Specific details are given in the following description to provide a thorough understanding of the embodiments. However, it will be understood that the embodiments may be practiced without these specific details. For example, circuits, systems, networks, processes, and other components may be shown as components in block diagram form in order not to obscure the embodiments in unnecessary detail. In other instances, well-known circuits, processes, algorithms, structures, and techniques may be shown without unnecessary detail in order to avoid obscuring the embodiments.

What is claimed is:

1. A method for managing a treatment for a subject diagnosed with neovascular age-related macular degeneration (nAMD), the method comprising:
receiving spectral domain optical coherence tomography (SD-OCT) imaging data of a retina of the subject;
extracting retinal feature data for a plurality of retinal features using the SD-OCT imaging data, the plurality of retinal features being associated with at least one of a set of retinal fluids or a set of retinal layers;
sending input data formed using the retinal feature data for the plurality of retinal features into a first machine learning model; and
predicting, via the first machine learning model, a treatment level for an anti-vascular endothelial growth factor (anti-VEGF) treatment to be administered to the subject based on the input data.

2. The method of claim 1, wherein the retinal feature data includes a value associated with a corresponding retinal fluid of the set of retinal fluids, the value selected from a group consisting of a volume, a height, and a width of the corresponding retinal fluid.

3. The method of claim 1 or 2, wherein the retinal feature data includes a value for a corresponding retinal layer of the set of retinal layers, the value selected from a group consisting of a minimum thickness, a maximum thickness, and an average thickness of the corresponding retinal layer.

4. The method of any one of claims 1-3, wherein a retinal fluid of the set of retinal fluids is selected from a group consisting of an intraretinal fluid (IRF), a subretinal fluid (SRF), a fluid associated with pigment epithelial detachment (PED), or a subretinal hyperreflective material (SHRM).

5. The method of any one of claims 1-4, wherein a retinal layer of the set of retinal layers is selected from a group consisting of an internal limiting membrane (ILM) layer, an outer plexiform layer-Henle fiber layer (OPL-HFL), an inner boundary-retinal pigment epithelial detachment (IB-RPE), an outer boundary-retinal pigment epithelial detachment (OB-RPE), or a Bruch's membrane (BM).

6. The method of any one of claims 1-5, further comprising:
forming the input data using the retinal feature data for the plurality of retinal features and clinical data for a set of clinical features, the set of clinical features including at least one of a best corrected visual acuity, a pulse, a diastolic blood pressure, or a systolic blood pressure.

7. The method of any one of claims 1-6, wherein predicting the treatment level comprises predicting a classification for the treatment level as either a high or a low treatment level.

8. The method of claim 7, wherein the high treatment level indicates sixteen or more injections of the anti-VEGF treatment during a selected time period after an initial phase of treatment.

9. The method of claim 7, wherein the low treatment level indicates five or fewer injections of the anti-VEGF treatment during a selected time period after an initial phase of treatment.

10. The method of any one of claims 1-9, wherein the extracting comprises:
extracting the retinal feature data for the plurality of retinal features from segmented images generated using a second machine learning model that automatically segments the SD-OCT imaging data, wherein the plurality of retinal features is associated with at least one of a set of retinal fluid segments or a set of retinal layer segments identified in the segmented images.

11. The method of claim 10, wherein the second machine learning model comprises a deep learning model.

12. The method of any one of claims 1-11, wherein the first machine learning model comprises an Extreme Gradient Boosting (XGBoost) algorithm.

13. The method of any one of claims 1-12, wherein the plurality of retinal features includes at least one feature associated with subretinal fluid (SRF) and at least one feature associated with pigment epithelial detachment (PED).

14. The method of any one of claims 1-13, wherein the SD-OCT imaging data comprises an SD-OCT image captured during a single clinical visit.

15. A method for managing an anti-vascular endothelial growth factor (anti-VEGF) treatment for a subject diagnosed with neovascular age-related macular degeneration (nAMD), the method comprising:
training a machine learning model using training input data to predict a treatment level for the anti-VEGF treatment, wherein the training input data is formed using training optical coherence tomography (OCT) imaging data;
receiving input data for the trained machine learning model, the input data comprising retinal feature data for a plurality of retinal features; and
predicting, via the trained machine learning model, the treatment level for the anti-VEGF treatment to be administered to the subject using the input data.

16. The method of claim 15, further comprising:
generating the input data using the training OCT imaging data and a deep learning model, wherein the deep learning model is used to automatically segment the training OCT imaging data to form segmented images and wherein the retinal feature data is extracted from the segmented images.

17. The method of claim 15 or 16, wherein the machine learning model is trained to predict a classification for the treatment level as either a high treatment level or a low treatment level, wherein the high treatment level indicates sixteen or more injections of the anti-VEGF treatment during a selected time period after an initial phase of treatment.

18. The method of claim 15 or 16, wherein the machine learning model is trained to predict a classification for the treatment level as either a high treatment level or a not high treatment level, wherein the high treatment level indicates six or more injections of the anti-VEGF treatment during a selected time period after an initial phase of treatment.

19. A system for managing an anti-vascular endothelial growth factor (anti-VEGF) treatment for a subject diagnosed with neovascular age-related macular degeneration (nAMD), the system comprising:
a memory containing machine readable medium comprising machine executable code; and
a processor coupled to the memory, the processor configured to execute the machine executable code to cause the processor to:
receive spectral domain optical coherence tomography (SD-OCT) imaging data of a retina of the subject;
extract retinal feature data for a plurality of retinal features using the SD-OCT imaging data, the plurality of retinal features being associated with at least one of a set of retinal fluids or a set of retinal layers;
send input data formed using the retinal feature data for the plurality of retinal features into a first machine learning model; and
predict, via the first machine learning model, a treatment level for an anti-vascular endothelial growth factor (anti-VEGF) treatment to be administered to the subject based on the input data.

20. The system of claim 19, wherein the machine executable code further causes the processor to extract the retinal feature data for the plurality of retinal features from segmented images generated using a second machine learning model that automatically segments the SD-OCT imaging data, wherein the plurality of retinal features is associated with at least one of a set of retinal fluid segments or a set of retinal layer segments identified in the segmented images.
